(19) United States
(12) Patent Application Publication
(10) Pub. No.: US 2005/0196316 A1
(43) Pub. Date: Sep. 8, 2005

(54) Vacuum line sanitization device and method

(76) Inventor: John J. Kochevar, Visalia, CA (US)

Correspondence Address: Richard A. Ryan, 8497 N. Millbrook Ave., suite 110, Fresno, CA, 93720, US

(21) Appl. No.: 10/792,044

(22) Filed: Mar. 2, 2004

(51) Int. Cl. A61L 2/20

(52) U.S. Cl. 422/33; 422/292

(57) ABSTRACT

A device and method for cleaning and sanitizing vacuum lines used in dental, medical and laboratory facilities comprising a canister body having an inlet and an outlet in communication with a fluid chamber inside the canister body. An inner chamber inside the canister body is in fluid communication with the fluid chamber and is suitable for receiving a supply of chemicals therein. The inlet is suitable for communication with a fluid supply line. Fluid from the supply line interacts with the chemicals in the fluid chamber to form a sanitizing mixture. One end of a vacuum line connects to the outlet to vacuum the sanitizing mixture through the vacuum line to clean and sanitize the vacuum line. To reduce chemical storage requirements, spillage and over-use, the chemical can be supplied in a cartridge form that dissolves, either fully or partially, when combined with the fluid inside the fluid chamber.

### CROSS-REFERENCE TO RELATED APPLICATIONS

The subject matter described herein was a subject of U.S. patent application Ser. No. 09/412,237 filed Oct. 5, 1999, now abandoned, which claimed the benefit of U.S. Provisional Patent Application No. 60/103,175 filed Oct. 6, 1998.

### BACKGROUND OF THE INVENTION

A. Field of the Invention

The field of the present invention relates generally to devices for sanitizing fluid system lines. More specifically, this invention relates to fluid system sanitizing devices for use in sanitizing the vacuum lines utilized in medical and dental facilities.

B. Background

Medical and dental offices, as well as hospitals and other patient treating facilities and laboratories, utilize a number of pieces of equipment which connect to flow lines that are part of a vacuum system. Medical offices and hospitals utilize vacuum systems to draw away blood and other fluids that accumulate in bodily cavities undergoing medical procedures. Laboratories utilize vacuum systems in their analysis of organic specimens. Dentists utilize a vacuum device connected to a vacuum system to draw away fluids (i.e., saliva, blood and rinse water) that accumulate in the patient's mouth during cleaning, drilling or other dental procedures. In general, the aspiration/vacuum systems utilized by medical and dental offices and laboratories contain a number of sterile and non-sterile pieces of equipment, including the flow lines connecting the equipment.

One well known problem with the above-mentioned vacuum systems is that the bodily fluids or organic materials flowing through the vacuum lines contaminate those lines. Even though the lines are connected to a vacuum system, there is always the potential that fluids left in the line can contaminate a subsequent patient or organic sample. Because of concerns with transmittal of diseases which are borne in human saliva and blood and with sample contamination, it is imperative that the vacuum lines be thoroughly cleaned and sanitized between patients or between samples. To address the problem of vacuum line contamination, various medical and dental organizations, as well as certain governmental agencies, have set specific cleaning requirements and recommendations for sanitizing vacuum lines.

The current method of sanitizing a vacuum line generally utilized by medical and dental offices is to place a chemical mixture containing chemicals which are capable of killing viruses and bacteria in an open sink or bucket. The chemical mixture is then sucked through the vacuum line to a discharge point (usually the sewer) until the sink or bucket is emptied or sufficient amount of the chemical mixture has been sucked through the vacuum line to properly sanitize the line. The vacuum line is then flushed with a rinse solution, such as a saline solution. The above-described vacuum line sanitization method has a number of significant problems. For instance, the typical sanitization method requires the medical or dental office to store and handle (i.e., when placing the chemicals into the sink or bucket for subsequent suction through the vacuum line) relatively dangerous chemicals. Due to the nature of the sanitization operation, there is a potential for over-use or under-use of the proper amount of chemicals. In addition, this procedure also requires a relatively significant amount of time for the technician in charge of cleaning and sanitizing the vacuum line to properly perform his or her duties. To save costs, many medical and dental offices order their chemical requirements in bulk, which further increases the risk of spillage during storage and/or handling of the large containers. Due to the chemicals and time involved, the costs associated with this treatment can be somewhat significant and can lead to some resistence to following the required cleaning procedures.

While the current method of cleaning and sanitizing vacuum lines in dental and medical offices is generally sufficient for cleaning and sanitizing the vacuum lines, there exists a need for a device that reduces the amount of effort required, reduces the need for storage of chemicals and reduces the likelihood of spillage while handling the subject chemicals. More specifically, there exists a need for a device that allows a dental or medical office employee to quickly and effectively clean and sanitize vacuum lines without the need to store large quantities of chemicals or risk spillage of such chemicals during the cleaning/sanitizing process.

### SUMMARY OF THE INVENTION

The vacuum line cleaning device of the present invention solves the problems identified above. Specifically, the present invention discloses a device for safely, easily and less expensively cleaning vacuum and other lines utilized in medical and dental facilities and laboratories that are subject to multiple frequent use to carry medical, biological and organic materials. The present invention allows the technician or other person in charge of cleaning and sanitizing vacuum lines to clean those lines without having to mix and use dangerous chemicals in open containers. In addition, the present invention provides a device that substantially reduces the facility's chemical storage requirements and provides a more consistent and reliable cleaning solution. The present invention will make it easier and less difficult for medical and dental offices, as well as other facilities, to prevent inter-contamination of patients and organic samples when using non-sterile aspiration units.

In one aspect of the present invention, the vacuum line sanitization device has a canister body having a sealable inlet and a controlled outlet, a non-drip nozzle attached to the outlet, an inner chamber inside the canister body and a chemical cartridge that is placed inside the inner chamber. In the preferred embodiment of the present invention, the components are lightweight and easy to connect to a variety of vacuum line conveying systems. The chemical composition of the cartridge utilized in the present invention should be odorless, non-toxic and non-foaming and be able to clean, disinfect and de-odorize all known non-sterile aspiration units. The subject device can be provided with a switch to manually start the cleaning process or be provided with an automated control mechanism for pre-set automatic cleaning of vacuum lines.

The present invention is easily installed by the users of the device without major modification to existing aspiration units. Once installed, the device is able to clean, disinfect and de-odorize a vacuum line system in only a few seconds of operation. Due to its lower cost, ease of use and effectiveness, the device will further promote the proper cleaning and decontamination of these lines between patients or samples. Compared to the current methods of cleaning vacuum lines, the chemical delivery system of the present invention virtually eliminates the likelihood of chemical spillage and over-use and substantially reduces the labor costs.

In operation, a chemical cartridge is placed inside the inner chamber of the canister body of the present invention by releasing the disconnect valve from the active water supply line and removing the twist nut allowing access to the chemical chamber. The addition of water or other fluid into the canister body automatically activates the chemicals in the chemical cartridge. One end of the vacuum line to be cleaned is attached to the outlet nozzle. After waiting sufficient time for the chemical reaction to take place, the suction system is activated and chemicals from the canister are sucked through the vacuum line and into a disposal system (i.e., the sewer). The amount of time to clean and sanitize a vacuum line, which can be preset at the time of installation of the device, will typically vary between five and ten seconds, depending upon the length of the vacuum line. If a preset amount of time is selected, it should be chosen for the longest line used in the vacuum system. Maintenance of the present invention simply consists of periodic replacement of the chemical cartridge inside the canister body.

Accordingly, the primary objective of the present invention is to provide a vacuum line sanitization device and method that provides the advantages discussed above and that overcomes the disadvantages and limitations associated with presently available vacuum line sanitization devices and methods.

It is also an important objective of the present invention to provide a device for cleaning fluid lines that enables such lines to be easily, quickly and relatively inexpensively cleaned, disinfected and deodorized.

It is also an important objective of the present invention to provide a device that utilizes a replaceable chemical cartridge that is suitable for multiple activation by the addition of a fluid into the device and is capable of cleaning, disinfecting and deodorizing fluid lines utilized in medical and dental facilities and laboratories.

It is also an important objective of the present invention to provide a device that comprises a lightweight canister having an inlet, an outlet and fluid chamber, wherein the outlet is suitable for connecting to a vacuum line and delivering a prescribed amount of chemicals from within the canister's fluid chamber through the vacuum line.

It is another objective of the present invention to provide a method for easily, quickly and relatively inexpensively cleaning fluid lines used in medical and dental offices and laboratories.

The above and other objectives of the present invention will be explained in greater detail by reference to the attached figures and the description of the preferred embodiment which follows. As set forth herein, the present invention resides in the novel features of form, construction, mode of operation and combination of parts presently described and understood by the claims.

### BRIEF DESCRIPTION OF THE DRAWINGS

In the drawings which illustrate the best modes presently contemplated for carrying out the present invention:

FIG. 1 is a cross-sectional side view of the vacuum line sanitization device of the present invention;

FIG. 2 is an end view of the inlet side of the device of the present invention;

FIG. 3 is an end view of the outlet side of the device of the present invention;

FIG. 4 is a side view of a chemical cartridge for use in the present invention;

FIG. 5 is a side view of an alternative embodiment for the vacuum line sanitization device of the present invention;

FIG. 6 is an end view of the inlet side of the alternative device shown in FIG. 5; and

FIG. 7 is an end view of the outlet side of the alternative device shown in FIG. 5.

### DETAILED DESCRIPTION

With reference to the figures where like elements have been given like numerical designations to facilitate the reader's understanding of the present invention, and particularly with reference to the embodiments of the vacuum line sanitization device of the present invention illustrated in the figures, various preferred embodiments of the present invention are set forth below. The enclosed description and drawings are merely illustrative of preferred embodiments and represent several different ways of configuring the present invention. Although specific components, materials, configurations and uses of the present invention are illustrated and set forth in this disclosure, it should be understood that a number of variations to the components and to the configuration of those components described herein and in the accompanying figures can be made without changing the scope and function of the invention set forth herein.

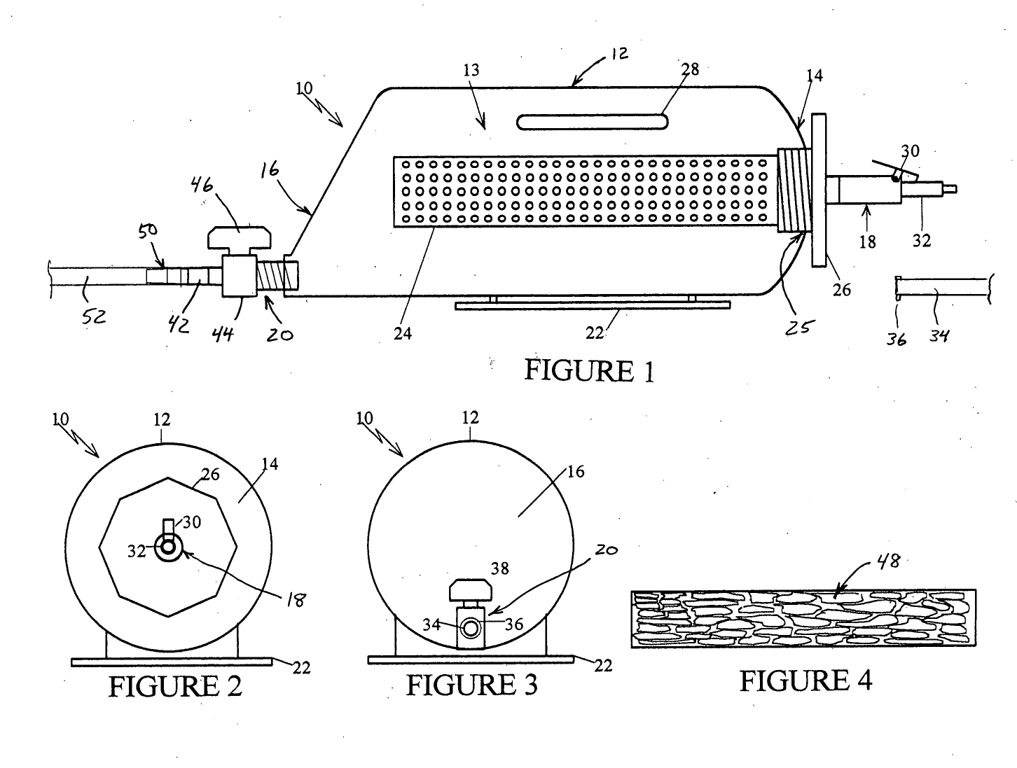

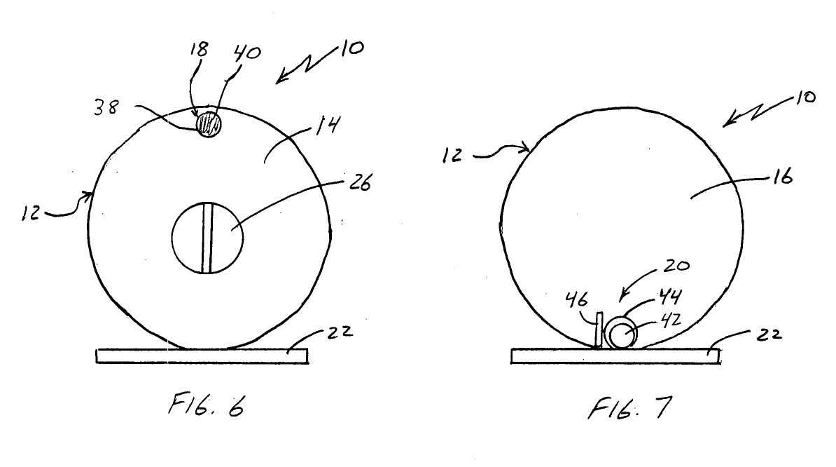

With particular reference to FIGS. 1 and 5, the vacuum line sanitization device of the present invention, designated generally as 10 in the figures, primarily comprises canister body 12, forming a main fluid chamber 13 therein, having a first end 14 and an opposing second end 16. As shown in FIGS. 1-3 and 5-7, at first end 14 is inlet 18 and at second end 16 is outlet 20. In the embodiment shown in the drawings, canister body 12 is attached to base 22. Inside canister body 12 is inner chamber 24 in fluid flow communication with main fluid chamber 13. Inner chamber 24 opens at chamber opening 25 at first end 14 of canister body 12 and is sealably closed with inner chamber cover 26, which is shown as being threadably connected to canister body 12. In the preferred embodiment, canister body 12 further comprises a fluid level indicator 28, which can be a glass or plastic see-through bubble indicator that allows the user to see inside of main fluid chamber 13.

In a preferred embodiment, canister body 12 is a generally hollow, cylindrically shaped member that is manufactured out of any number of lightweight, sturdy and corrosion resistant materials, such as aluminum, stainless steel and other metals, or a variety of plastics, composites or other suitable materials. Depending on the material chosen for canister body 12, fluid chamber 13 may require a suitable lining, coating or otherwise be configured so material used for canister body 12 does not interact with the chemicals used to sanitize the vacuum line. In a preferred embodiment, canister body 12 is approximately the size and shape of a soda can having flat ends. Although shown in FIG. 1 in its preferred cylindrical shape, canister body 12 can be made into any number of shapes. Canister body 12 can attach to base 22 using glue, tape or any other suitable adhesive, or canister body 12 can be made integral with base 22 as a single structure. If used, base 22 should be suitable for setting the vacuum line sanitization device 10 on a relatively flat surface, such as a table or counter top. Base 22 could also include a mechanism for attaching base 22 to the flat surface, such as double tape, screws or clamps.

In the preferred embodiment, inlet 18 comprises an inlet valve 30 at first end 14 having a male adapter 32 configured to attach to a pressurized water supply line 34 having a female-configured receptacle 36. In this embodiment, inlet valve is of the type known as a quick-disconnect valve having a ¼″ line adaptability. Inlet valve 30 is configured to prevent flow of fluid from inside fluid chamber 13 out inlet 18. Male adapter 32 of inlet valve 30, once inserted into the water supply line, will allow continuous filling until such time when inlet valve 30 is released from water supply line 34 at male adapter 32 to allow removal or opening of inner chamber cover 26. In an alternative embodiment, shown in FIGS. 5 through 7, inlet 18 has an inlet valve 30 comprising sealable opening 38 at first end 14 having seat valve 40 located therein. Seat valve 40 can be of the type known as a rubber bushing wherein a rubber disk having a small slit is placed in opening 38 in a manner that keeps the slit closed until penetrated by an object and then forces the slit closed when the object is removed. This type of sealable opening 38 is commonly used with various medical and dental devices. Opening 38 and seat valve 40 should be sized so as to fit water supply tools, such as a water aspirator used at the end of a water line, typically found in most medical and dental offices and laboratories. In one embodiment, opening 38 has an inside diameter of ⅜″ to fit most such tools. When closed, seat valve 40 should be suitable for preventing any fluid from inside canister body 12 from exiting out opening 38 during filling and normal use of device 10.

In the preferred embodiment, outlet 20 comprises an outlet stem 42 in fluid communication with the main fluid chamber 13 inside canister body 12 through second end 16. Outlet stem 42 has a closeable valve 44 thereon, which can be of the thumb screw type (as shown) that enables the user to slightly twist valve wheel 46 to allow the sanitizing mixture within main fluid chamber 13 to flow out outlet stem 42. Outlet stem 42 should be sized and configured to fit the typical vacuum lines found in medical and dental offices. In the preferred embodiment, stem 42 is configured to fit vacuum lines having a ⅜″ outside diameter.

In the preferred embodiment, inner chamber 24 comprises a perforated sleeve member, as best shown in FIG. 1, that is suitable for holding a supply of chemicals, such as a chemical cartridge 48, and allowing fluid flow communication between chemical cartridge 48 and fluid chamber 13. Chemical cartridge 48 should be sized to fit through chamber opening 25, which is closed with inner chamber cover 26, and be placed within inner chamber 24. To place chemical cartridge 48 inside inner chamber 24, the operator of the device first releases inlet valve 30 and removes inner chamber cover 26 from first end 14 of canister body 12 to expose chamber opening 25. One such type of cover 26 is shown in FIGS. 1 and 2 and FIGS. 5 and 6 as a screw-type cap that removably attaches to first end 14 to sealably close chamber opening 25 to inner chamber 24. After placing chemical cartridge 48 inside inner chamber 24, the operator would close off inner chamber 24 and seal canister body 12 by reattaching inner chamber cover 26 to chamber opening 26 at first end 14.

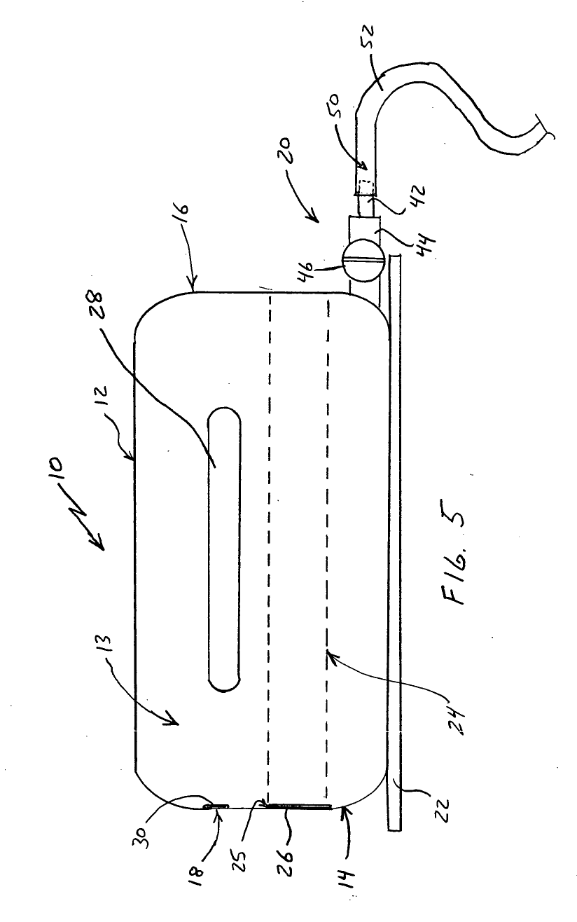

Chemical cartridge 48 should comprise a combination of chemicals suitable for cleaning, disinfecting and deodorizing fluid lines after the lines are used for transporting human, biological or organic materials. A number of chemical mixtures are currently available that can clean and sanitize vacuum lines as set forth with this invention. Such chemical mixtures include those utilizing sodium phosphate and other sanitizing agents. In use with the preferred embodiment of the present invention, the chemicals are provided in a solid form, as shown in FIG. 4, such that the operator only has to release inlet valve 30, open inner chamber cover 26 and place the chemical cartridge 48 inside inner chamber 24, thereby virtually eliminating the spillage and handling problems common with presently available sanitization devices. Naturally, if provided in solid form, the chemical cartridge 40 must be sized and configured to fit within inner chamber 24. The chemical mixture, preferably, should be suitable for having a long “shelf life” and for allowing multiple uses (i.e., multiple filling of canister body 12 with fluid) of the same cartridge 48. The preferred chemicals should be non-gaseous, not contain chlorine, be odor free, non-toxic and suitable for regulated release of chemical solution, as well as effective for cleaning and sanitizing vacuum lines used in dental, medical and laboratory facilities.

The chemicals in chemical cartridge 48, when placed inside inner chamber 24, are activated by the water (the preferred activating agent), or other appropriate fluid, flowing from fluid supply line 34 through inlet 18 into canister body 12. The fluid should be selected such that it forms a sanitizing mixture when combined with the supply of chemicals (i.e., the chemical cartridge 48). In a preferred embodiment, the canister body 12 would hold approximately twelve to sixteen ounces of fluid. After the chemical cartridge 48 is placed inside inner chamber 24 and inner chamber cover 26 is re-attached, fluid is filled up to the fluid level indicator 28. As known to those skilled in the art, fluid level indicator 28 should be located above the level of inner chamber 24 to ensure sufficient fluid is placed inside fluid chamber 13 to allow chemical cartridge 48 to fully react with the fluid.

In use, chemical cartridge 48 is placed inside inner chamber 24 in fluid chamber 13 and inner chamber cover 26 is installed on canister body 12 to sealably close inner chamber 24. The operator connects first end 50 of vacuum line 52 to outlet stem 42 at outlet 20 and also connects the opposite end of vacuum line 52 (not shown) to the vacuum system (not shown). The discharge of the vacuum system is configured such that the sanitizing mixture will drain into an appropriate drainage system (i.e., the sewer) after passing through vacuum line 52. In the preferred embodiment, water supply line 34 is continuously connected to inlet valve 30 so that it is able to supply fluid to vacuum line sanitization device 10 of the present invention and, therefore, sanitize vacuum line 52 when necessary or desired (i.e., on demand). Alternatively, water supply line 34 can be selectively connected to device 10 to sanitize vacuum line 52 when necessary or desired. In the alternative embodiment, the end of fluid supply line 34 is inserted through seat valve 40 at sealable opening 38 to allow fluid flow into fluid chamber 13 when necessary or desired. In either configuration, the operator initiates fluid flow in fluid supply line 34 to allow fluid to flow into fluid chamber 13 until the level of the fluid is at or above fluid indicator level 28, at which time the operator terminates the fluid flow to device 10. Chemical cartridge 48 is allowed time to sufficiently dissolve in order to produce the sanitizing fluid (this may be partial dissolving or complete dissolving, depending on the chemical mixture and form utilized) that will be used to clean and sanitize vacuum line 52. The operator turns valve wheel 46 to open valve 44 and then activates the vacuum system to vacuum the dissolved liquid chemical from fluid chamber 13 through vacuum line 52. Valve 36 remains open until all the sanitizing mixture in fluid chamber 13 is vacuumed out, allowing the dissolved chemicals to flow through and thereby clean, disinfect and deodorize vacuum line 52 (typically this will take five to ten seconds). After the chemical solution has cleaned vacuum line 52, the operator turns valve wheel 46 to close valve 44.

Although the preferred embodiment is shown and described as a relatively small unit, the device 10 of the present invention is not so limited. In the preferred embodiment, base 22 can sit on or against a flat surface such as a counter or wall. The device can also be installed under sinks, which are commonly found in most dental and medical offices and laboratories, and connected to one or more vacuum lines and/or systems. Furthermore, the locations of inlet 18, outlet 20 and opening for inner chamber 24 are not limited to the places indicated on the referenced figures. For example, inlet 18 and outlet 20 can be on the same end of body 12. Also, device 10 can utilize a single inlet or opening for both the activating fluid and for the chemical if desired. A single inlet or opening may be desirable if the chemical is provided in a granular or liquid form, or if the chemical is to be pre-mixed prior to being placed inside fluid chamber 13.

In addition to the above, device 10 can be further provided with a mechanism for indicating when chemical cartridge 48 in inner chamber 24 needs to be replaced. Two such mechanisms for indicating when it is necessary to replace chemical cartridge 48 or other chemicals are the use of color (i.e., the loss of color of the mixture) to indicate lack of or reduction in chemical activity or an electronic diode in communication with the mixture that sends a visual or audible signal to the user when chemical activity reduces to a predetermined level.

While there is shown and described herein certain specific alternative forms of the invention, it will be readily apparent to those skilled in the art that the invention is not so limited, but is susceptible to various modifications and rearrangements in design and materials without departing from the spirit and scope of the invention. In particular, it should be noted that the present invention is subject to modification with regard to the dimensional relationships set forth herein and modifications in assembly, materials, size, shape, and use.

1. A vacuum line sanitization device for sanitizing a vacuum line, comprising:
a canister body having a first end and an opposing second end, said canister body forming a fluid chamber therein, said fluid chamber configured to receive a supply of chemicals suitable for sanitizing the vacuum line;
an inlet on said canister body in communication with said fluid chamber for disposing a fluid inside said fluid chamber, said fluid selected so as to form a sanitizing mixture when combined with said supply of chemicals;
means for sealing said inlet for preventing flow of said fluid from said fluid chamber out of said canister body through said fluid inlet; and
an outlet on said canister body in communication with said fluid chamber, said outlet comprising means for connecting said outlet to one end of the vacuum line, said outlet further comprising means for selectively dispensing said fluid through the vacuum line so as to clean and sanitize the vacuum line,
whereby the vacuum line is sanitized by opening said dispensing means to draw said sanitizing mixture through the vacuum line.

2. The vacuum line sanitization device according to claim 1 further comprising an inner chamber in said canister body, said inner chamber in fluid communication with said fluid chamber, said inner chamber configured to receive said supply of chemicals.

3. The vacuum line sanitization device according to claim 2, wherein said inner chamber comprises a perforated sleeve.

4. The vacuum line sanitization device according to claim 1, wherein said inlet is at said first end of said canister body and said outlet is at said second end of said canister body.

5. The vacuum line sanitization device according to claim 1, wherein said connecting means comprises an outlet stem sized and configured to be received into one end of the vacuum line.

6. The vacuum line sanitization device according to claim 1, wherein said sealing means is a quick-release valve.

7. The vacuum line sanitization device according to claim 1, wherein said sealing means is a seat valve.

8. The vacuum line sanitization device according to claim 1, wherein said dispensing means comprises a closeable valve.

9. The vacuum line sanitization device according to claim 1, wherein said supply of chemicals is a solid chemical cartridge.

10. The vacuum line sanitization device according to claim 1 further comprising an indicator configured to indicate when said supply of chemicals must be replaced.

11. A vacuum line sanitization device for sanitizing a vacuum line, comprising:
a canister body having a first end and an opposing second end, said canister body forming a fluid chamber therein, said fluid chamber configured to receive a supply of chemicals suitable for sanitizing the vacuum line;
an inlet on said canister body in communication with said fluid chamber for disposing a fluid inside said fluid chamber, said fluid selected so as to form a sanitizing mixture when combined with said supply of chemicals;
an inlet valve at said inlet, said inlet valve configured to connect to a fluid supply line and receive said fluid therefrom, said inlet valve further configured to prevent flow of said fluid from said fluid chamber out of said canister body through said fluid inlet; and
an outlet on said canister body in communication with said fluid chamber, said outlet having an outlet stem and an outlet valve, said outlet stem sized and configured to connect said outlet to an end of the vacuum line, said outlet valve configured to selectively allow said fluid to flow through the vacuum line so as to clean and sanitize the vacuum line,
whereby the vacuum line is sanitized by opening said outlet valve to draw said sanitizing mixture through the vacuum line.

12. The vacuum line sanitization device according to claim 11 further comprising an inner chamber in said canister body, said inner chamber in fluid communication with said fluid chamber, said inner chamber configured to receive said supply of chemicals.

13. The vacuum line sanitization device according to claim 12, wherein said inner chamber comprises a perforated sleeve.

14. The vacuum line sanitization device according to claim 11, wherein said inlet valve comprises a quick-release valve.

15. The vacuum line sanitization device according to claim 11, wherein said inlet valve comprises a seat valve.

16. The vacuum line sanitization device according to claim 11, wherein said supply of chemicals is a solid chemical cartridge.

17. The vacuum line sanitization device according to claim 16 further comprising an inner chamber in said canister body, said inner chamber in fluid communication with said fluid chamber, said inner chamber configured to receive said solid chemical cartridge.

18. The vacuum line sanitization device according to claim 11 further comprising an indicator configured to indicate when said supply of chemicals must be replaced.

19. A method of sanitizing a vacuum line connected to a vacuum system, comprising the steps of:
placing a supply of chemicals inside a fluid chamber disposed within a canister body of a sanitization device;
sealing said canister body;
connecting a fluid supply line to an inlet valve on said canister body and connecting an end of the vacuum line to an outlet on said canister body, said outlet having an outlet valve thereon in fluid communication with said fluid chamber;
disposing a quantity of a fluid into said fluid chamber through said inlet valve;
waiting for said fluid to chemically react with said supply of chemicals to form a sanitizing mixture in said fluid chamber;
opening said outlet valve; and
activating the vacuum system so as to initiate flow of said sanitizing mixture from said fluid chamber to clean and sanitize the vacuum line.

20. The method according to claim 19, wherein said sanitization device further comprises an inner chamber in said canister body, said inner chamber in fluid communication with said fluid chamber, said inner chamber configured to receive said supply of chemicals.
